(19) United States
(12) Patent Application Publication
(10) Pub. No.: US 2023/0337988 A1
(43) Pub. Date: Oct. 26, 2023

(54) AI-BASED DETECTION OF PHYSIOLOGIC EVENTS USING AMBULATORY ELECTROGRAMS

(71) Applicant: Cardiac Pacemakers, Inc., St. Paul, MN (US)

(72) Inventors: Ramesh Wariar, Blaine, MN (US); Viktoria A. Averina, Shoreview, MN (US); Deepa Mahajan, North Oaks, MN (US); Keith R. Maile, New Brighton, MN (US); Bin Mi, Arden Hills, MN (US); Craig Stolen, New Brighton, MN (US); Scott R. Vanderlinde, Plymouth, MN (US)

(21) Appl. No.: 18/136,194

(22) Filed: Apr. 18, 2023

Related U.S. Application Data
(60) Provisional application No. 63/334,239, filed on Apr. 25, 2022.

(51) Int. Cl. A61B 5/00

(52) U.S. Cl. CPC A61B 5/7267; A61B 5/7278

(57) ABSTRACT

Systems and methods for detecting a physiological event or estimating a physiological parameter using ambulatory electrograms of a subject are discussed. An exemplary system includes a computing device that can receive ambulatory electrograms collected by an ambulatory medical device (AMD) associated with a subject, and apply the ambulatory electrograms to a trained machine learning model to estimate a physiological parameter or to detect a physiological event in the subject. The same or a different machine learning model can be trained to detect an operating status of the AMD using the ambulatory electrograms. The system comprises an output device to output the estimated physiological parameter, the detected physiological event, or the detected device operating status a user or a process such as to initiate or titrate a therapy.

### CLAIM OF PRIORITY

This application claims the benefit of U.S. patent application Ser. No. 63/334,239, filed on Apr. 25, 2022, which is hereby incorporated by reference in its entirety.

### TECHNICAL FIELD

This document relates generally to medical devices, and more particularly, to systems, devices and methods for artificial intelligence (AI)-based detection of physiological events using ambulatory electrograms.

### BACKGROUND

Ambulatory medical devices (AMD), such as implantable medical devices (IMDs), have been used for monitoring patient health condition or disease states and delivering therapies. For example, implantable cardioverter-defibrillators (ICDs) may be used to monitor for certain abnormal heart rhythms and to deliver electrical energy to the heart to correct the abnormal rhythms. Some IMDs may be used to monitor for chronic worsening of cardiac hemodynamic performance, such as due to congestive heart failure (CHF), and to provide cardiac stimulation therapies, including cardiac resynchronization therapy (CRT) to correct cardiac dyssynchrony within a ventricle or between ventricles.

Some AMDs have sensors that are capable of sensing physiological information from a patient, such as electrocardiograms (ECGs) or electrograms (EGMs) representing cardiac electrical activities. For example, a Holter monitor is a wearable device with skin electrodes to monitor ambulatory ECG. ECG has been used to diagnose and monitor various cardiac conditions or diseases, such as cardiac arrhythmias, coronary heart disease, heart attacks, cardiomyopathy, left ventricular systolic dysfunction, among others. The ECG has also been used to investigate symptoms such as chest pain, palpitations, dizziness, and shortness breach, among other heart-related problems.

### OVERVIEW

Artificial intelligence (AI) can be used to interpret ECG and to assist in detecting cardiac diseases such as cardia arrhythmias. Machine learning (ML) models that mimic the function of brain neurons using a series of interconnected computational statistical algorithms or nodes can be trained on a large data set of ECGs to recognize a particular cardiac disease or diagnosis. AI-based analysis of ECGs may also provide valuable insights into some non-cardiac conditions and seemingly unrelated or indirect biological parameters such as cardiac ejection fraction or blood potassium.

Electrocardiograms are typically measured using a 12-lead system in a clinical setting. This may be inconvenient for some patients especially those who require continuous or frequent ECG monitoring for an extended period of time. Ambulatory monitoring via devices such as implantable cardiac devices, insertable cardiac monitors (ICM), subcutaneous ICDs (S-ICDs), wearable or holdable devices, among other ambulatory monitors, may also allow for ECG or EGM (e.g., intracardiac or epicardiac EGMs) data collection, analysis, and detection of cardiac events. Compared to the in-clinic 12-lead ECG, wearable or implantable devices can acquire ambulatory ECGs or EGMs with greater frequency or even continuously without the changes of electrode configurations typically seen in a clinic. The ambulatory ECGs or EGMs can be analyzed to produce repeated measurements of physiological parameters. The measurements can be trended over time, and used to provide more accurate early detection or prediction of cardiac or other physiological events.

The present disclosure describes AI-based systems, devices, and methods for estimating physiological parameters or detecting physiological events in a subject using ambulatory EGMs. In accordance with one embodiment, a system includes a computing device configured to receive ambulatory EGMs collected by an AMD worn or implanted in the body of the subject, and to apply the received ambulatory EGMs to a trained machine learning (ML) model to estimate a physiological parameter or to detect a physiological event in the subject. The trained ML model is trained using a training dataset comprising EGMs collected from a patient population and assessments of physiological parameters or physiological events in the patient population. In some examples, the same or a different ML model can be trained to detect, from the ambulatory EGMs of the subject, an operating status of the AMD. The estimated physiological parameter, the detected physiological event, or the detected operating status of the AMD can be provided to a user, or to a process such as to initiate or titrate a therapy. In some examples, the estimated physiological parameter, when satisfying a condition, can be used to trigger a sensor to directly measure the physiological parameter. The direct measurement can be used to adjust the trained ML model to improve its performance of parameter estimation.

Example 1 is a system for detecting physiological events in a subject, the system comprising: a computing device configured to: receive ambulatory electrograms of the subject collected by an ambulatory medical device (AMD); and apply the received ambulatory electrograms to a trained machine learning model to estimate a physiological parameter or to detect a physiological event in the subject; and an output unit configured to output the estimated physiological parameter or the detected physiological event to a user or a process.

In Example 2, the subject matter of Example 1 optionally includes the computing device that can be further configured to: determine or confirm a physical characteristic of at least one sensor using the ambulatory electrograms; and detect a physiological event or estimate a physiological parameter in the subject using the ambulatory electrograms and the determined or confirmed physical characteristic of the at least one sensor, wherein to detect the physiological event, the computing device is configured to apply the received ambulatory electrograms to a trained machine learning model to estimate the physiological parameter or to detect the physiological event in the subject using the ambulatory electrograms sensed by the at least one sensor, and wherein the physical characteristic is a sensor type or a form factor of the at least one sensor.

In Example 3, the subject matter of Example 2 optionally includes the computing device that can include a training module configured to generate the trained machine learning model, including: constructing a training dataset including ambulatory electrograms collected from a patient population and assessments of physiological parameters or physiological events in the patient population; and training a machine learning model using the constructed training dataset until a convergence or training stopping criterion is satisfied, the trained machine learning model representing a correspondence between the ambulatory electrograms of the patient population and the physiological parameters or the physiological events in the patient population.

In Example 4, the subject matter of Example 3 optionally includes the at least one sensor that can be configured to sense from the subject physiological information different than the ambulatory electrogram, and wherein the training module is configured to train the machine learning model using a deep learning algorithm comprising a deep neural network.

In Example 5, the subject matter of any one or more of Examples 2-4 optionally includes the computing device that can be configured to apply the received ambulatory electrograms to the trained machine learning model to estimate the physiological parameter including at least one of: a cardiac parameter; a respiratory parameter; a circulating biomarker; or a systemic or local fluid status.

In Example 6, the subject matter of any one or more of Examples 2-5 optionally includes the computing device that can be configured to apply the received ambulatory electrograms to the trained machine learning model to detect the physiological event including at least one of: a cardiac arrhythmia; a worsening heart failure event; a heart failure comorbidity condition; a neurological condition; or a response to medication.

In Example 7, the subject matter of any one or more of Examples 2-6 optionally includes the computing device that can be further configured to apply the received ambulatory electrograms to the trained machine learning model to detect an operating status of the AMD.

In Example 8, the subject matter of Example 7 optionally includes the operating status of the AMD that indicate at least one of: a change in position, posture, or orientation of the AMD; or a change in an device-tissue interface the AMD.

In Example 9, the subject matter of any one or more of Examples 1-8 optionally includes the AMD that can be configured to collect the ambulatory electrograms of the subject continuously or periodically via one or more attachable or implantable electrodes.

In Example 10, the subject matter of any one or more of Examples 1-9 optionally includes the AMD that can include at least one of: an insertable cardiac monitor; a subcutaneous implantable cardioverter-defibrillator; or a wearable or holdable cardiac monitor.

In Example 11, the subject matter of any one or more of Examples 1-10 optionally includes the AMD that can include a therapy circuit configured to initiate or adjust a therapy to the subject based on the estimated physiological parameter or the detected physiological event.

In Example 12, the subject matter of any one or more of Examples 2-11 optionally includes the computing device that can be configured to: apply the received ambulatory electrograms to the trained machine learning model to estimate the physiological parameter; and in response to the estimated physiological parameter satisfying a condition, trigger at least one of the at least one sensor to directly measure the physiological parameter.

In Example 13, the subject matter of Example 12 optionally includes the computing device that can include a calibration circuit configured to adjust the trained machine learning model based at least on the directly measured physiological parameter.

In Example 14, the subject matter of any one or more of Examples 1-13 optionally includes the computing device that can be configured to perform parallel computing to estimate multiple physiological parameters or to detect multiple physiological events substantially concurrently.

In Example 15, the subject matter of Example 14 optionally includes the computing device that can include multiple processors or a multi-core processor comprising multiple computing units, each of the multiple processors or the multiple computing units configured to apply a portion of the ambulatory electrograms of the subject to a respectively trained machine learning model to estimate a respective physiological parameter or to detect a respective physiological event in the subject.

Example 16 is a method for detecting physiological events in a subject, the method comprising: receiving ambulatory electrograms of the subject collected by an ambulatory medical device (AMD); determining, via a computing device, a form factor of at least one sensor using the ambulatory electrograms, the at least one sensor configured to sense from the subject physiological information different than the ambulatory electrograms; serially detecting a physiological event or estimating a physiological parameter in the subject using the ambulatory electrograms and the physiological information sensed by the at least one sensor; and providing the estimated physiological parameter or the detected physiological event to a user or a process.

In Example 17, the subject matter of Example 16 optionally includes serially detecting the physiological event that can include applying the received ambulatory electrograms to a trained machine learning model to estimate the physiological parameter or to detect the physiological event in the subject.

In Example 18, the subject matter of Example 17 optionally includes: constructing a training dataset including ambulatory electrograms collected from a patient population and assessments of physiological parameters or physiological events in the patient population; and training a machine learning model using the constructed training dataset until a convergence or training stopping criterion is satisfied, the trained machine learning model representing a correspondence between the ambulatory electrograms of the patient population and the physiological parameters or the physiological events in the patient population.

In Example 19, the subject matter of any one or more of Examples 17-18 optionally includes applying the received ambulatory electrograms to the trained machine learning model and detecting an operating status of the AMD.

In Example 20, the subject matter of any one or more of Examples 16-19 optionally includes initiating or adjusting a therapy via the AMD to the subject based on the estimated physiological parameter or the detected physiological event.

In Example 21, the subject matter of any one or more of Examples 16-20 optionally includes, in response to the estimated physiological parameter satisfying a condition: triggering direct measurement of the physiological parameter using at least one of the at least one sensor; and adjusting the trained machine learning model based at least on the direct measurement of the physiological parameter.

This Overview is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates an example system including an ambulatory medical device configured to sense or detect physiological information from a patient via one or more sensors.

FIG. 3 is a block diagram illustrating a portion of an exemplary AI-based physiological event detection system configured to detect a physiological event or to estimate a physiological parameter using ambulatory EGMs.

FIGS. 4A-4B are diagrams illustrating an example of training a machine learning (ML) model and using the trained ML model to estimate a physiological parameter, to detect a physiological event, or to detect a device operating status.

FIG. 5 is a flowchart illustrating an example of a method for detecting a physiological event or to estimate a physiological parameter using ambulatory EGMs.

FIG. 6 illustrates generally a block diagram of an example machine upon which any one or more of the techniques (e.g., methodologies) discussed herein may perform.

### DETAILED DESCRIPTION

Disclosed herein are AI-based systems, devices, and methods for detecting a physiological event or estimating a physiological parameter using ambulatory EGMs. An exemplary system includes a computing device configured to receive ambulatory EGMs collected by an ambulator medical device (AMD) associated with a subject, and apply the ambulatory EGMs to a trained machine learning (ML) model to estimate a physiological parameter or to detect a physiological event in the subject. The same or a different machine learning model can be trained to detect an operating status of the AMD using the ambulatory electrograms. The system comprises an output device to output the estimated physiological parameter, the detected physiological event, or the detected device operating status to a user or a process such as to initiate or titrate a therapy.

The systems, devices, and methods discussed in this document may improve prediction or early detection of various physiological events, such as cardiovascular or respiratory disorders, systemic or local fluid status, circulating biomarkers, or neurological disorders. Although alternative solutions such as direct measurement using application-specific sensors can provide desired detection performance, operation of the application-specific sensors are generally associated with higher system complexity and implementation cost, and some sensors may not be sensitive enough to provide early indications of an event. The present disclosure describes early physiological event detection using ambulatory ECGs or EGMs, which can be acquired rapidly using lower cost, simpler, or less obtrusive systems, apparatus, and methods. The availability of a large volume of ECG or EGM data makes them suitable for AI-based application, including training and validating an ML model for estimating physiological parameters and detecting physiological events. Early detection of physiological events may be used to pre-screen patients for further evaluation using, for example, more sophisticated and expensive tests, or for medical intervention or therapy optimization (e.g., drug titration or timing, etc.).

In certain examples, the systems, apparatus, and methods discussed herein can provide additional use for existing sensors, reduce sensor cost, enable earlier intervention, improve patient outcomes, and reduce overall medical system costs. The systems and methods described herein, in certain examples, represent an improved form of physiological event detection and patient intervention over existing techniques. In certain examples, patients can be monitored, and the patient, caregiver, clinician, or one or more other system or user can be alerted to a change in patient condition. In other examples, the systems and methods described herein can provide intervention or therapy optimization recommendation, or can directly provide or alter a therapy to the patient.

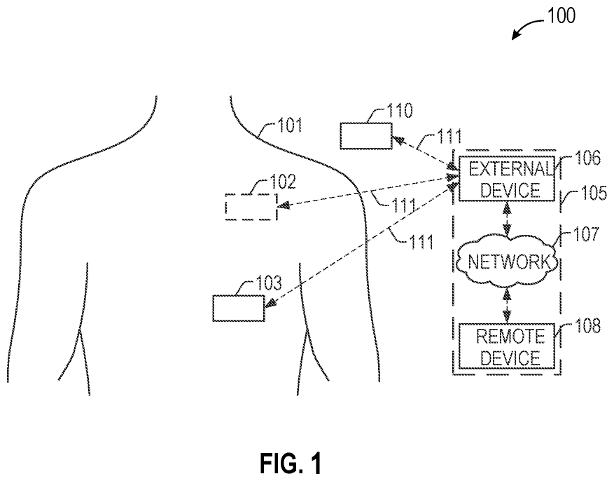

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more ambulatory medical devices, an external system 105, and a communication link 111 providing for communication between the one or more ambulatory medical devices and the external system 105. The one or more ambulatory medical devices can include an implantable medical device (IMD) 102, a wearable medical device 103, or one or more other implantable, leadless, subcutaneous, external, wearable, or ambulatory medical devices configured to monitor, sense, or detect information from, determine physiological information about, or provide one or more therapies to treat various conditions of the patient 101, such as one or more cardiac or non-cardiac conditions (e.g., dehydration, sleep disordered breathing, etc.).

In an example, the implantable medical device 102 can include one or more traditional cardiac rhythm management devices implanted in a chest of a patient, such as pacemakers, pacemaker/defibrillators, cardiac resynchronization therapy (CRT) devices, cardiac remodeling control therapy (RCT) devices, among others. The implantable medical device 102 can have a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. By way of example and not limitation, the lead system may be transvenously inserted into, or positioned on a surface of, a portion of the heart such as a right atrium (RA), a right ventricle (RV), a left atrium (LA), or a left ventricle (LV), or any tissue between or near the heart portions. In another example, the implantable medical device 102 can include a monitor implanted, for example, subcutaneously in the chest of patient 101. Examples of such subcutaneous devices can include insertable cardiac monitors (ICM), subcutaneous ICDs (S-ICDs), among others. The implantable medical device 102 including a housing containing circuitry and, in certain examples, one or more sensors, such as a temperature sensor, an accelerometer for sensing heart sounds, etc.

The implantable medical device 102 can include sensing circuitry configured to sense cardiac electrical activity using the electrodes on the lead system. The sensed cardiac electrical activity is generally referred to as electrograms (EGMs). In an example, the sensing circuit can sense the EGMs continuously or periodically as specified frequency. The implantable medical device 102 can further include an assessment circuit configured to detect or determine specific physiological information of the patient 101, or to determine one or more physiological conditions, or provide information or an alert to a user, such as the patient 101 (e.g., a patient), a clinician, or one or more other caregivers or processes.

The implantable medical device 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the implantable medical device 102 or one or more of the other ambulatory medical devices, etc. In some examples, therapy can include cardiac resynchronization therapy for rectifying dyssynchrony and improving cardiac function in heart failure patients. In other examples, the implantable medical device 102 can include a drug delivery system, such as a drug infusion pump to deliver drugs to the patient for managing arrhythmias or complications from arrhythmias, hypertension, or one or more other physiologic conditions. In other examples, the implantable medical device 102 can include one or more electrodes configured to stimulate the nervous system of the patient or to provide stimulation to the muscles of the patient airway, etc.

The wearable medical device 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.). The wearable medical device 103 can include sensing circuitry coupled on one or more electrodes or sensors. In an example, the sensing circuitry of the wearable medical device 103 can be configured to sense cardiac electrical activity, such as ambulatory ECGs, via electrodes electrically coupled to the sensing circuitry.

The external system 105 can include a dedicated hardware/software system, such as a programmer, a remote server-based patient management system, or alternatively a system defined predominantly by software running on a standard personal computer. The external system 105 can manage the patient 101 through the implantable medical device 102 or one or more other ambulatory medical devices connected to the external system 105 via a communication link 111. In other examples, the implantable medical device 102 can be connected to the wearable medical device 103, or the wearable medical device 103 can be connected to the external system 105, via the communication link 111. This can include, for example, programming the implantable medical device 102 to perform one or more of acquiring physiologic data, performing at least one self-diagnostic test (such as for a device operating status), analyzing the physiologic data, or optionally delivering or adjusting a therapy for the patient 101. Additionally, the external system 105 can send information to, or receive information from, the implantable medical device 102 or the wearable medical device 103 via the communication link 111. Examples of the information can include real-time or stored physiologic data from the patient 101, diagnostic data, such as detection of patient hydration status, hospitalizations, responses to therapies delivered to the patient 101, or device operating status of the implantable medical device 102 or the wearable medical device 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can estimate physiological parameters, detect a physiological event, or detect an operating status of the IMD 102 or the wearable medical device 103 using information collected from the IMD 102 or the wearable medical device 103, such as ambulatory EGMs or ambulatory ECGs collected from the patient 101. In various examples, artificial intelligence (AI) or machine learning (ML) can be used to assist in estimating physiological parameters, or detecting physiological events or device operating status. For example, one or more of the external device 106 or the remote device 108 can include an ML engine that uses a trained ML model to assess and identify different physiological events or physiological parameters. In some examples, one or more of the external device 106 or the remote device 108 can include a computing platform utilizing a parallel processing with interconnected processing nodes and queues that form a workflow for estimating multiple physiological parameters or detecting multiple physiological events substantially concurrently.

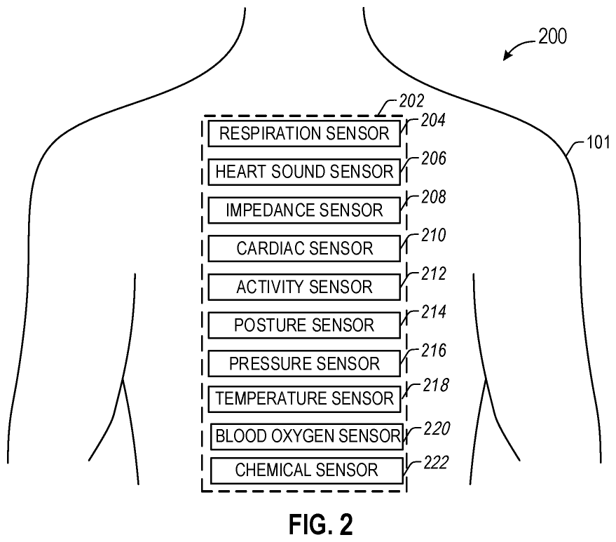

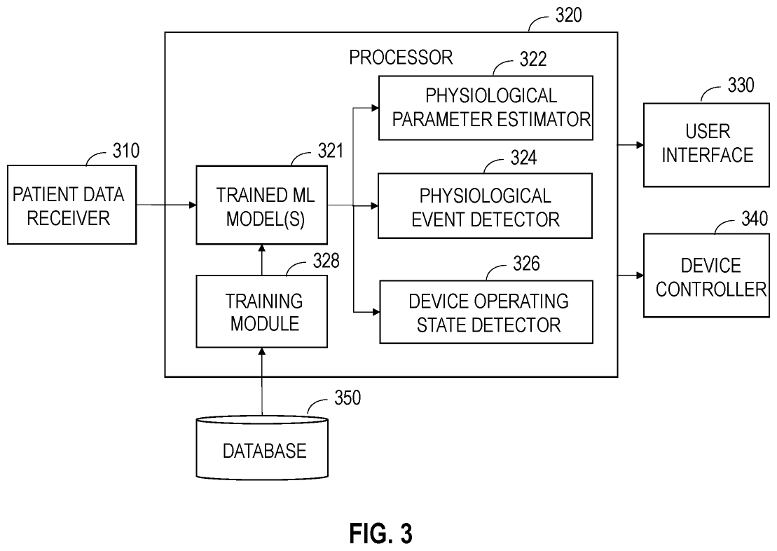

In some examples, the IMD 102 or the wearable medical device 103 may collect other sensor signals different than the ambulatory ECGs or EGMs, as described below with reference to FIG. 2. Such sensors may have different physical characteristics, such as different form factors or sensor types. For example, different sensors may have form factors suitable for wearing on different body parts (e.g., helmet, glasses, earpieces, necklace, chest band, belt, wristwatch, bracelet, gloves, ring, ankle band, shoes, socks, detachable patches, or garment), or for implanting at different body locations. In addition, specific sensors designed for specific locations or general use can have different profiles, surface areas, or forms that can impact the received signals in different ways. Such sensor signals may also be used to estimate physiological parameters, detect physiological events, or detect a device operating status. In an example, physiological events can be serially detected using ambulatory ECGs or EGMs, and one or more other sensors with respective types and form factors. The serial detection may involve estimating a physiological parameter or generating an initial detection of a physiological event using ambulatory ECGs or EGMs, and when the estimated physiological parameter or the initial detection of the physiological event satisfies a specific condition, triggering one or more other application-specific sensors to directly measure the physiological parameter or to detect the physiological event. The ECG- or EGM-based parameter estimation event detection can be confirmed, rejected, or modified by the direct measurement or detection by the one or more application-specific sensors. In some examples, the direct measurement or detection by the application-specific sensors can be used to calibrate the ECG- or EGM-based physiological parameter estimation or physiological event detection. Examples of AI-based physiological parameter estimation and physiological event detection using ambulatory EGMs are discussed below with reference to FIG. 3.

One or more of the external device 106 or the remote device 108 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

FIG. 2 illustrates an example system 200 including an ambulatory medical device (AMD) 202 configured to sense or detect physiological information from a patient 101 via one or more sensors. The AMD 202 can be a single device, or a plurality of medical devices or monitors. The AMD 202 can be an example of one or more of the implantable medical device 102 or the wearable medical device 103, and configured to estimate physiological parameters, detect physiological events, or detect a device operating status using ambulatory physiological information of a patient.

The AMD 202 can include one or more physiological sensors configured to sense respective physiological information of a patient 101. Examples of the physiological information can include cardiac or respiratory parameters, circulating biomarkers, systemic or local fluid status, neurological conditions, and patient responses to medication or other treatment regimens, among others. The AMD 202 may include circuitry, or a microprocessor, that can estimate physiological parameters, detect physiological events, or detect a device operating status using at least some of the sensor data, such as ambulatory ECGs or EGMs collected by the cardiac sensor 210. In an example of AI-based physiological parameter estimation and physiological event detection system, the sensor data can be used to establish a training dataset for training and validating an ML model. In some examples, a ML model trained for estimating a physiological parameter using ECGs or EGMs can be updated or refined using direct measurements of the physiological parameter by an application-specific sensors, as described further below with reference to FIG. 3.

By way of example and not limitation, the sensors can include one or more of a respiration sensor 204, a heart sound sensor 206, an impedance sensor 208, a cardiac sensor 210, an activity sensor 212, a posture sensor 214, a pressure sensor 216, a temperature sensor 218, a blood oxygen sensor 220, or a chemical sensor 222. The respiration sensor 204 can be configured to receive respiration information, including but not limited to a respiration rate (RR), a respiration volume (tidal volume), an RSBI, indicators of dyspnea, tachypnea, etc. Such respiration information may be used to detect respiratory events such as pulmonary edema, dyspnea, and pneumonitis, among others.

The heart sound sensor 206 may take a form of an accelerometer or a microphone sensor, and can be configured to receive heart sound information, including but not limited to intensity (e.g., amplitudes) one or more of S1, S2, S3, or S4 heart sound components, and timing parameters such as systolic timing intervals measured using the heart sound components. Such heart sounds information may be used to detect deterioration in cardiac function (e.g., reduced contractility, reduced ejection fraction, reduced systolic blood pressure, increased end-diastolic volume, and reduced cardiac output), diastolic abnormalities, myocardial depression, ischemia and microvascular dysfunction, cardiac arrhythmias, and WHF, among others. For example, a decrease in S1 amplitude may indicate a reduced myocardial contractility, a decrease in S2 amplitude may indicate a reduced arterial blood pressure and cardiac output, and an increase in S3, S4, or S4/S3 ratio may indicate worsened diastolic function.

The microphone or accelerometer sensor may sense physiological information other than heart sounds. In an example, a microphone may be used to sense information of patient voice, which can be further analyzed to detect patient signs or symptoms associated with a particular physiological event. The microphone sensor may sense respiration such as respiratory rate, which may be used to detect tachypnea or other disordered breathing. An accelerometer sensor may be used to sense physical activity which may indicate fatigue, or neurologic disorders such as tremor, altered gait, or seizure, or to sense body shaking which may indicate rigor.

The impedance sensor 208 can be configured to receive impedance information (e.g., intracardiac, intrathoracic, or transthoracic impedance). A decrease in body impedance may be indicative of body fluid accumulation, such as in the lungs known as pulmonary edema. In an example, the impedance sensor 208 may detect pulmonary edema based on a decrease in thoracic impedance. In some examples, the impedance sensor 208 may detect an increase in body impedance indicative of dehydration.

The cardiac sensor 210 can be configured to receive cardiac electrical information, such as ECGs or EGMs as described above with reference to FIG. 1. The ECGs or EGMs may be used to detect cardiac parameters or cardiac events such as heart rate, heart rate variability, cardiac synchrony, or various cardiac arrhythmias.

The activity sensor 212 can be configured to receive information about a physical motion (e.g., activity, steps, etc.), and the posture sensor 214 can be configured to receive posture or position information. Changes in physical activity or changes in posture may be indicative of development or worsening of heart failure. The physical activity or motion information and the posture or position information may additionally or alternatively be used to trigger one or more other physiologic sensors, such as heart sounds, impedance, or pressure data acquired under a specified physical activity level or a specified posture.

The pressure sensor 216 may be configured to receive pressure information. In an example, the pressure sensor 216 is a blood pressure sensor configured to sense blood pressure, which may further be used to detect hypotension. In another example, the pressure sensor 216 is configured to sense abdominal pressure.

The temperature sensor 218 may be configured to receive body temperature information. Examples of the body temperature sensor may include a thermal couple, a thermistor, an infrared sensor, or a temperature sense integrated circuit. As fever is one of adverse systemic inflammatory responses to various medical conditions, a high body temperature (e.g., above 38° C.), along with other sensor information, may be used to detect a physiological event, or to calibrate a physiological event detector based on other physiological information such as ambulatory ECGs or EGMs.

The blood oxygen sensor 220 may be configured to receive information about blood oxygen saturation. In an example, the blood oxygen sensor 220 is a pulse oximeter. Patient with certain medical conditions may develop hypoxia, a condition where the body or a body region is deprived of adequate oxygen supply at the tissue level. A low arterial blood oxygen saturation (e.g., less than 92%), along with other sensor information, may be used to detect a physiological event, or to calibrate a physiological event detector based on other physiological information such as ambulatory ECGs or EGMs.

The chemical sensor 222 may be configured to receive information of one or more blood chemicals or circulating biomarkers. Circulating biomarkers are generally nucleic acids, extracellular vesicles, proteins and metabolites from all metastatic sites as well as normal organ physiologic turn over or impact of systemic drug treatment. Examples of the biomarkers can include plasma glucose; biomarkers of kidney injury such as blood urea nitrogen (BUN) and creatinine; cardiac biomarkers such as natriuretic peptides, myoglobin, troponin and creatine kinase, among others. Circulating biomarkers may additionally include blood electrolyte levels such as potassium, sodium, or calcium levels. Data from the chemical sensors 222 can be used to detect various types of physiological events such as cardiac arrhythmias, heart failure, renal dysfunctions, to control conditions such as electrolyte imbalance such as hyper- or hypokalemia (abnormally high or low blood potassium level), or hyper- or hypoglycemia (abnormally high or low blood glucose level), either as intrinsic physiologic reactions or in response to medical treatment (e.g., side effects, inappropriate treatment, or inadequate dosage).

FIG. 3 is a block diagram illustrating a portion of an exemplary AI-based physiological event detection system 300 that can be configured to detect a physiological event or to estimate a physiological parameter using patient data such as ambulatory ECGs or EGMs. At least a part of the system 300 can be implemented in one or more of the implantable medical device 102, the wearable medical device 103, or the AMD 202. Additionally or alternatively, at least a part of the system 300 can be implemented in a device in a remote patient management system, such as the external device 106 or the remote device 108.

The system 300 can include a patient data receive 310, a processor 320, a user interface 330, and a device controller 340. In some examples, the system 300 can include or be communicatively coupled to a database 350. The patient data receiver 310 can be configured to receive patient information, such as physiological information of a patient (or a group of patients) from one or more of the physiologic sensors, such as sensors 204-222 as discussed above with reference to FIG. 2. In an example, the patient data receiver 310 can be included in a wearable or implantable device, and collect ambulatory patient data, such as ambulatory ECGs or EGMs, continuously or periodically or at a specified frequency such as daily, weekly, biweekly, or monthly.

The processor 320 can include one or more of a physiological parameter estimator 322, a physiological event detector 324, or a device operating state detector 326. The physiological parameter estimator 322 can estimate a physiological parameter using at least a portion of the receive patient information, such as ambulatory ECGs or EGMs. In an example, the estimated physiological parameter can include one or more cardiac parameters, such as ECG parameters (e.g., QT prolongation, ST segment elevation indicative of acute myocardial infarction, T wave inversion, heart rate, heart rate variability, premature atrial contractions (PACs), premature ventricular contractions (PVCs), cardiac timing intervals, etc.). The cardiac parameters may also include cardiac systolic parameters (e.g., left ventricular ejection fraction, right ventricular ejection fraction, stroke volume, cardiac wall thickness indicative of hypertrophy, cardiac wall motion or change of wall motion, ejection time, pre-ejection period, etc.), cardiac diastolic parameters such as obtained from transthoracic Doppler echocardiographic analysis (e.g., E and A peak velocities and their ratio E/A, E-wave deceleration time, Isovolumic relaxation time), and valvular function parameters (e.g., stenosis or regurgitation associated with one or more of aortic, mitral, pulmonary, or tricuspid values). In another example, the estimated physiological parameter can include one or more respiratory parameters, such as a indicators of chronic obstructive pulmonary disease (COPD), central sleep apnea, or obstructive sleep apnea, among other respiratory disorders. In another example, the estimated physiological parameter can include a systemic or local fluid status, such as fluid accumulation in lungs or other organs or tissues. Fluid overload is related to increased mortality and also lead to several complications like pulmonary edema. In yet another example, the estimated physiological parameter can include one or more circulating biomarkers, such as plasma glucose levels, various kidney injury biomarkers, cardia arrhythmia and heart failure biomarkers, or blood electrolyte levels.

The physiological event detector 324 can detect a physiological event using at least a portion of the receive patient information, such as ambulatory ECGs or EGMs. In some examples, the physiological event detector 324 can detect the physiological event using the estimated physiological parameter provided by the physiological parameter estimator 322. The detection physiological event can include, for example, a cardiac arrhythmia, such as atrial fibrillation (AF), atrial flutter, supra-ventricular tachycardia, ventricular tachycardia, ventricular fibrillation, Wolff-Parkinson-White (WPW) syndrome, reentry arrhythmia, among others. In an example, the detected physiological event can include an arrhythmia burden indicating frequency or time spent on a particular type of arrhythmia, such as an AF burden represented by a proportion (e.g., percentage) of time an individual is in AF during a monitoring period. In an example, the detected physiological event can include a worsening heart failure (WHF) event, or a heart failure comorbidity such as renal insufficiency, diabetes mellitus, chronic obstructive pulmonary disease (COPD), sleeping disorders like obstructive and central sleep apnea, anemia, liver diseases such as cirrhosis or other diseases of volume regulation. In another example, the detected physiological event can include a neurological condition, such as an sympathovagal imbalance syndrome, orthostatic hypotension, pain, migraine, and emotional disorder, among others. In another example, the detected physiological event can include patient response to medication or other treatment regimens, such as side effects experienced by patients. In yet another example, the detected physiological event can include cardiotoxicity or cardiopulmonary toxicity of immunotherapy, which can be represented by deterioration in cardiac function (e.g., reduced contractility, reduced ejection fraction, reduced systolic blood pressure, increased end-diastolic volume, and reduced cardiac output), diastolic abnormalities, myocardial depression, ischemia and microvascular dysfunction, cardiac arrhythmias, and WHF, among others.

The device operating state detector 326 can detect an operating status of an ambulatory device, such as one or more of the implantable medical device 102, the wearable medical device 103, or the AMD 202. In an example, the device operating status can include a change in position, posture, or orientation of an ambulatory medical device on the subject, such as flipping of an implantable device (e.g., the implantable medical device 102) in the skin pocket, or a twiddler syndrome associated with unintentional or deliberate manipulation of the implantable device within its skin pocket by the patient. The flipping or twiddler of the implantable device can cause coiling of the lead or its dislodgement, resulting in failure of providing therapy or inappropriately delivered therapy. In another example, the device operating status can include a change in the device-tissue interface, such as air presence in the skin pocket for the implantable device. In yet another example, the device operating status can include a state of its operating environment, such as presence of noise or interferences or co-implant of another device.

In some examples, the patient data receiver 310 can collect ambulatory patient data, such as ambulatory ECGs or EGMs, continuously or periodically. Accordingly, one or more of the physiological parameter estimator 322, the physiological event detector 324, or the device operating state detector 326 can perform their respective tasks continuously or periodically at specific frequency such as daily, weekly, biweekly, or monthly. In some examples, the patient data receiver 310 can collect ambulatory patient data at variable frequencies, such as changing from daily to weekly to biweekly then to monthly. The serially estimated parameters and detected events can be trended over time to provide more accurate assessment of disease progression and patient health.

One or more of the physiological parameter estimator 322, the physiological event detector 324, or the device operating state detector 326 can perform their respective tasks of parameter estimation or event detection based on an analysis of ambulatory ECGs or EGMs. Although the ambulatory ECGs or EGMs do not provide direct measurements of certain physiological parameters (e.g., respiratory parameters, circulating biomarkers, and certain cardiac parameters) that could otherwise be measured directly using application-specific sensors, operation of the application-specific sensors are generally associated with higher system complexity and implementation cost, and some sensors are not sensitive to provide early indications of an event. In contrast, ambulatory ECGs or EGMs can be acquired rapidly using lower cost, simpler, or less obtrusive systems, apparatus, and methods, and can provide an estimation of the physiological parameters and an earlier detection of physiological events or device operating status.

Artificial intelligence (AI) or machine learning (ML) can be used to improve ECG- or EGM-based physiological parameter estimation or physiological event detection. As illustrated in FIG. 3, one or more of the physiological parameter estimator 322, the physiological event detector 324, or the device operating state detector 326 can perform their respective tasks of parameter estimation or event detection using one or more trained machine learning (ML) model(s) 321. The trained ML model(s) 321 can have a neural network structure comprising an input layer, one or more hidden layers, and an output layer. Patient information received by the patient data receiver 310, such as ambulatory ECGs or EGMs, or trends of signal metrics of the ECGs or EGMs, can be provided to the input layer of the ML model(s) 321. The ML model(s) 321 can propagate the input data or data trends through one or more hidden layers to the output layer. The ML model(s) 321 can provide the system 300 with the ability to perform tasks, without explicitly being programmed, by making inferences based on patterns found in the analysis of data. The ML model(s) 321 explores the study and construction of algorithms (e.g., ML algorithms) that may learn from existing data and make predictions about new data. Such algorithms operate by building the ML model(s) 321 from training data in order to make data-driven predictions or decisions expressed as outputs or assessments.

The ML model(s) 321 may be trained using supervised learning or unsupervised learning. Supervised learning uses prior knowledge (e.g., examples that correlate inputs to outputs or outcomes) to learn the relationships between the inputs and the outputs. The goal of supervised learning is to learn a function that, given some training data, best approximates the relationship between the training inputs and outputs so that the ML model can implement the same relationships when given inputs to generate the corresponding outputs. Unsupervised learning is the training of an ML algorithm using information that is neither classified nor labeled, and allowing the algorithm to act on that information without guidance. Unsupervised learning is useful in exploratory analysis because it can automatically identify structure in data.

Some common tasks for unsupervised learning include clustering, representation learning, and density estimation. Some examples of commonly used unsupervised learning algorithms are K-means clustering, principal component analysis, and autoencoders. Some common tasks for supervised learning are classification problems and regression problems. Classification problems, also referred to as categorization problems, aim at classifying items into one of several category values. Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

In an example, the ML model can be trained using deep learning. The ML model has an architecture of a deep neural network Examples of DNN include a convolutional neural network (CNN), a recurrent neural network (RNN), a deep belief network (DBN), long-term and short-term memory (LSTM) network, a transfer learning network, or a hybrid neural network comprising two or more neural network models of different types or different model configurations.

Another type of ML is collaborative learning that trains an algorithm across multiple decentralized devices holding local data, without exchanging the data. This approach stands in contrast to traditional centralized machine learning techniques where all the local datasets are uploaded to one server, as well as to more classical decentralized approaches which often assume that local data samples are identically distributed. Collaborative learning enables multiple actors to build a common, robust machine learning model without sharing data, thus allowing to address critical issues such as data privacy, data security, data access rights and access to heterogeneous data.

As illustrated in FIG. 3, the trained ML model(s) 321 may be generated by a training module 328. The training module 328 can be included in the processor 320 as shown in FIG. 3, or alternatively be implemented in a separate unit. To train an ML model, a training dataset can be constructed using patient information collected from a patient population each having respective AMDs of the same or similar type (e.g., implantable devices or wearable devices). The information of the patient population can be stored in the database 350. The training data may include patient physiological data collected by their respective AMDs, and assessments of specific physiological parameters, physiological events, or operating status of the respective AMDs. The assessments can be provided by a user (e.g., a clinician), and include characterization of the physiological parameter or the detected events (e.g., onset, offset, duration, rate, rhythm, and cardiac timing and synchrony for a cardiac arrhythmic episode). In some examples, the assessment can be made based on diagnostic tests using application-specific sensors or instruments (e.g., echocardiograms or other diagnostic imaging tests, electrical mapping for diagnosing cardiac arrhythmias, invasive glucose test via finger prick, glucose strip, or continuous glucose monitors). The training data may additionally include patient demographics, medical history and health conditions, treatment and outcome, etc.

In an example of training a EGM-based ML model, the training data can include, among other patient information, EGMs or data trends of signal metrics generated from the EGMs collected from the patient population, and assessment of physiological parameters or identification of physiological events, which can be provided by human experts as output labels. The training of the ML model may be performed continuously or periodically, or in near real time as additional patient information are received by the patient data receiver 310. The training involves algorithmically adjusting one or more ML model parameters, until the ML model being trained satisfies a specified training convergence criterion, such as the model outputs being as close as possible (within a specific margin) to the output labels.

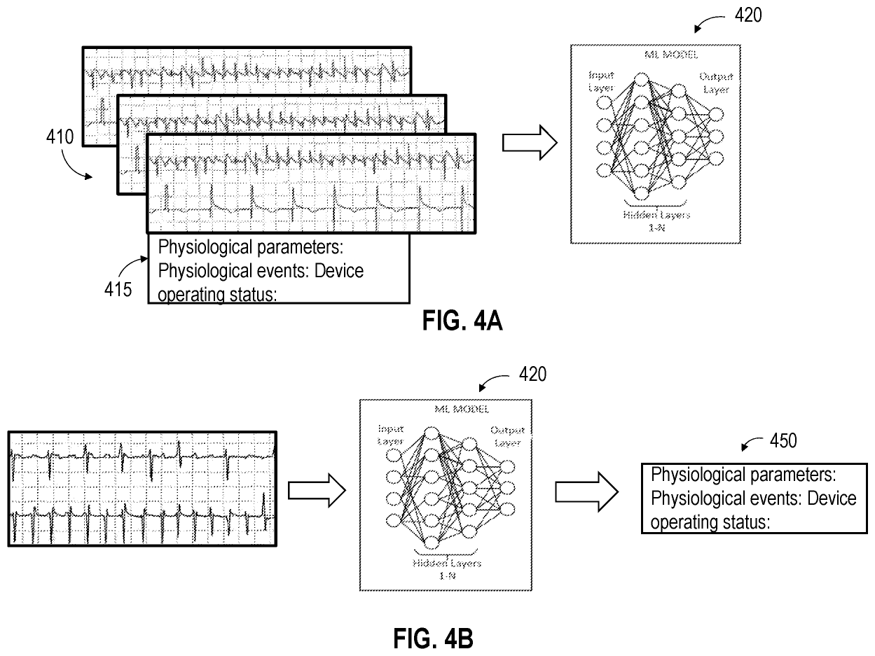

In some examples, a plurality of ML models can be separately trained and applied in different applications. For example, a first ML model (or a first set of ML models) may be trained to establish a correspondence between EGMs collected from a population (optionally along with other information) and assessments of physiological parameters. The physiological parameter estimator 322 can apply EGMs collected from a patient to the trained first ML model(s) to estimate one or more physiological parameters. In another example, a second ML model (or a second set of ML models) may be trained to establish a correspondence between EGMs collected from a population (optionally along with other information) and an assessment of physiological events. The physiological event detector 324 can apply EGMs sensed from a patient to the trained second ML model(s) to detect one or more physiological events. In another example, a third ML model (or a third set of ML models) may be trained to establish a correspondence between EGMs collected from a population (optionally along with other information) and an assessment of device operating status. The device operating state detector 326 can apply EGMs sensed from a patient to the trained third ML model(s) to detect a device operating status. In some examples, the trained third ML model(s) may detect device operating status (e.g., air pocket/tissue contact, or device flipping or twiddler) based on signal features extracted from the input EGMs, such as EGM noise or low amplitudes EGMs. Examples of training an ML model and using the trained ML model to estimate physiological parameters or to detect physiological events using patient EGMs are discussed below such as with reference to FIGS. 4A-4B.

In some examples, the processor 320 can be configured to perform parallel computing where multiple computing resources are used to perform respective computing tasks such as estimating multiple physiological parameters, or detecting multiple physiological events, substantially concurrently or simultaneously. In an example, the processor 320 can be a multi-core processor comprising multiple computing units each configured to apply a respective portion of the ambulatory EGMs of the subject to a respectively trained ML model to estimate a respective physiological parameter or to detect a respective physiological event in the subject. In some examples, multiple processors, each resembling the processor 320 or a portion thereof, can perform substantially concurrent computations such as estimating multiple physiological parameters, or detecting multiple physiological events.

The user interface 330 can include an output unit configured to output one or more of the estimated physiological parameter, the detected physiological event, or the detected device operating status to a user (e.g., a patient, a caregiver, or a clinician), such as to a display or one or more other user interface. The output can include a score, a trend, or other indication. The output may include recommendations for taking further diagnostic tests, consulting with caregiver, taking or adjusting medication, lifestyle management, comorbidity assessment, management or prevention of major adverse cardiovascular events and/or cardiovascular mortality, among other standards of medical care depending on the type of the physiological events. In other examples, the assessment circuit 304 can be configured to provide an output to another circuit, machine, or process.

The device controller 340 can control, adjust, or cease a therapy of a medical device, a drug delivery system, etc. based on the estimated physiological parameter, the detected physiological event, or the detected device operating status. In some examples, the device controller 340 can trigger an application-specific sensor to directly measure a physiological parameter when the estimated physiological parameter satisfies a specific condition. An application-specific sensor of a particular type and/or of a particular form factor can be determined or information about a specific sensor can be confirmed (e.g., a received sensor type or form can be confirmed using sensed information) based on the ECG- or EGM-based physiological parameter estimation or physiological event detection. Such determination or confirmation can improve system performance with different sensors, or enable the device controller to be used with different sensors or information from different sensors or sources. For example, if the trained ML model estimates, based on the input EGMs, an abnormal level of a biomarker (e.g., plasma glucose level), then a glucose sensor can be triggered to perform direct measurement of the glucose level. The ambulatory EGMs and the application-specific sensor signals can be collected continuously or periodically at respective frequencies, such as daily, weekly, biweekly, or monthly. In some examples, the ambulatory EGMs and the sensor signals can be collected at variable frequencies, such as changing from daily to weekly to biweekly then to monthly. The ECG- or EGM-based physiological parameter estimation or physiological event detection can be confirmed, rejected, or modified by the direct measurement or detection by the application-specific sensor. Such serial detection involving an ECG- or EGM-based initial parameter estimation and event detection followed by a confirmative detection using an application-specific sensor can improve the efficiency and accuracy of physiological event detection, reduce system complexity, and save system and implementation cost.

In some examples, the device controller 340 can use the direct measurements of physiological parameters by the application-specific sensors to calibrate one or more of the physiological parameter estimator 322, the physiological event detector 324, or the device operating state detector 326. For examples, if an trained ML model detects, based on the input EGMs, an abnormal kidney injury biomarker level, then direct measurement of kidney injury biomarker level can be made and used to adjust EGM acquisition (e.g., timing, sampling rate, duration) and analysis. In some examples, the direct measurements by the application-specific sensors can be used to update the trained ML model(s) 321 (e.g., by adjusting weights assigned to one or more nodes of the neural network), or to retrain the ML model(s) 321.

In some examples, the processor 320 can detect from the received patient data (e.g., ambulatory ECGs or EGMs) information about device interference from a co-implant or an external noise source. Examples of the device interference may include pacing pulses from a co-implant, electromagnetic noises, optical sensing noise, or accelerometer interferences, among others. Based on the detected interference information, the device controller 340 can calibrate one or more of the physiological parameter estimator 322, the physiological event detector 324, or the device operating state detector 326, or adjust the trained ML model(s) 321. In some examples, the device controller 340 can adjust the EGM sensing configuration or settings of other ambulatory sensors to avoid or reduce interference and improve signal quality.

Portions of the system 300, such as one or more of the processor 320 (or a portion thereof, such as one or more of the physiological parameter estimator 322, the physiological event detector 324, the device operating state detector 326, or the training module 328) or the device controller 340, may include respective circuit sets comprising one or more other circuits or sub-circuits. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Portions of the system 300, such as one or more of the processor 320 (or a portion thereof) or the device controller 340, may be implemented as a part of a microprocessor circuit. The microprocessor circuit may be a dedicated processor such as a digital signal processor, application specific integrated circuit (ASIC), microprocessor, or other type of processor for processing information including the physiologic signals received from the patient data receiver 310. Alternatively, the microprocessor circuit may be a general-purpose processor that may receive and execute a set of instructions of performing the functions, methods, or techniques described herein.

In some examples, portions of the system 300 can be implemented in a cloud-based computing platform (the “cloud”) that provides cloud-based services including, for example, data storage, computing services, and provisioning of customer services, among others. For example, the database 350 can reside in a cloud storage device. Additionally or alternatively, the cloud may provide computing services for one or more of the physiological parameter estimator 322, the physiological event detector 324, the device operating state detector 326, or the training module 328 to fulfil their respective objectives. An authorized user can remotely access the cloud storage device to retrieve physiological information from the database 350, or to use the computing services therein to perform data analysis such as estimating physiological parameters or detecting physiological events in a patient, or detecting an operating status of the AMD of the patient.

FIGS. 4A-4B are diagrams illustrating an example of training an EGM-based ML model, and using the trained model to estimate a physiological parameter, to detect a physiological event, or to detect a device operating status based on an input EGM sensed from a patient. FIG. 4A illustrates an ML model training (or learning) phase during which an ML model 420 may be trained using training data comprising EGMs 410 collected from a population. The training data may also include, for each of the EGMs, respective annotated data 415 comprising assessment of physiological parameters, physiological events, or device operating status for corresponding EGMs. The assessment can be provided by a user, such as based on direct measurements of physiological parameters by application-specific sensors, or diagnostic tests using application-specific instruments (e.g., echocardiograms or other diagnostic imaging tests, electrical mapping for diagnosing cardiac arrhythmias, invasive glucose test via finger prick, glucose strip, or continuous glucose monitors). The ML model 740 can be trained using supervised learning, unsupervised learning, or reinforcement leaning. Examples of ML model architectures and algorithms may include, for example, decision trees, neural networks, support vector machines, or a deep neural networks, etc. Examples of deep neural network can include a convolutional neural network (CNN), a recurrent neural network (RNN), a deep belief network (DBN), a long-term and short-term memory (LSTM) network, a transfer learning network, or a hybrid neural network comprising two or more neural network models of different types or different model configurations. The training of the ML model may be performed continuously or periodically, or in near real time as additional patient data are made available. The training involves algorithmically adjusting one or more ML model parameters, until the ML model being trained satisfies a specified training convergence criterion. The trained ML model 420 can establish a correspondence between EGMs and one or more of a physiological parameter, a physiological event, or a device operating status.

FIG. 4B illustrates an inference phase during which an EGM 430 sensed from a patient can be applied to the trained ML model 420 to automatically estimate one or more physiological parameters, to detect a physiological event, or to detect a device operating status. The estimated parameters and/or the detected events 450 can be provided to the user (e.g., a patient, a caregiver, or a clinician), such as to a display or one or more other user interface. Additionally or alternatively, the estimated parameters and/or the detected events 450 can be output to another circuit, machine, or process, such as to control, adjust, or cease a therapy of a medical device, a drug delivery system, etc. In some examples, the estimated parameters and/or the detected events 450 can be used to trigger direct measurement of a physiological parameter using an application-specific sensor, or to calibrate one or more of a physiological parameter estimator 322, a physiological event detector 324, or a device operating state detector 326, or to update the trained ML model 420 such as by adjusting weights assigned to one or more nodes of a neural network.

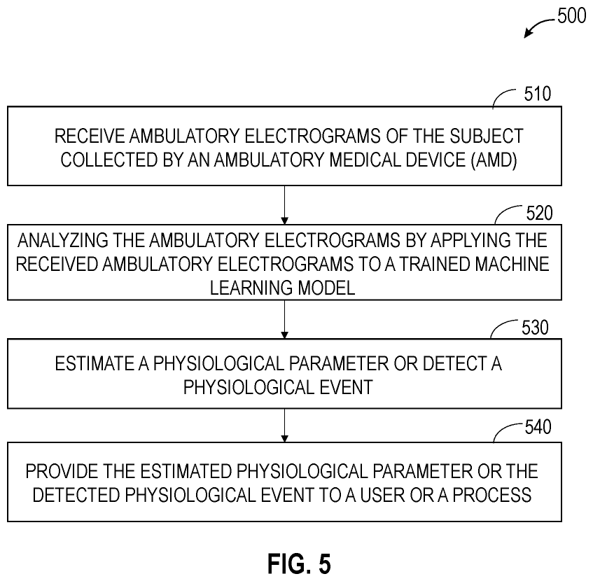

FIG. 5 is a flowchart illustrating an example of a method 500 for detecting a physiological event or to estimate a physiological parameter using patient information such as ambulatory electrograms (EGMs). The method 500 may be implemented in and executed by a computing device, such as one or more of the implantable medical device 102, the wearable medical device 103, or the AMD 202. Alternatively, the method 500 may be implemented in and executed by a device in a remote patient management system, such as the external device 106, the remote device 108, or a cloud-based computing platform.

The method 500 commences at 510, where physiological information may be received from the patient using an ambulatory medical device (AMD), such as the implantable medical device 102, the wearable medical device 103, or the AMD 202. Various physiological information can be received using respective sensors or electrodes, such as sensors 204-222 as discussed above with reference to FIG. 2. In an example, the received physiological information includes ambulatory EGMs. Ambulatory EGMs, optionally along with other physiological information, can be collected continuously or periodically at a specific frequency such as daily, weekly, biweekly, or monthly.

At 520, the received ambulatory EGMs can be analyzed using the computing device, or a portion thereof such as the processor 320 of the AI-based physiological event detection system 300. The analysis can include applying the received ambulatory EGMs to one or more trained machine learning (ML) models, such as the trained ML model(s) 321. As discussed above with respect to FIGS. 3 and 4A, the ML models may be trained using a training dataset including ambulatory EGMs collected from a patient population and assessments of physiological parameters or physiological events in the patient population. The ML model can be trained using supervised learning or unsupervised learning. In an example, the ML model can be trained using deep learning. The ML model has an architecture of a deep neural network (DNN). The training of the ML model may be performed continuously or periodically, or in near real time as additional patient information are received. The training involves algorithmically adjusting one or more ML model parameters, until the ML model being trained satisfies a specified training convergence criterion. The trained ML model represents a correspondence between the ambulatory EGMs of the patient population and the physiological parameters or the physiological events in the patient population. Such a trained ML model is also referred to as an EGM-based ML model.

At 530, a physiological parameter can be estimated, and/or or a physiological event can be detected, based on the analysis of the EGMs using the trained ML model(s). The physiological parameter estimation and the physiological event detection can be carried out using the physiological parameter estimator 322 and the physiological event detector 324, respectively, as discussed above with referenced to FIG. 3. Examples of the estimated physiological parameters can include a cardiac parameter, a respiratory parameter, a circulating biomarker, or a systemic or local fluid status, among others. Examples of the detected physiological event including a cardiac arrhythmia, a worsening heart failure event, a heart failure comorbidity condition, a neurological condition, or a response to medication, among others.

In some examples, at step 520, the ML model (or a separate ML model other than the ML model trained for estimating physiological parameters or for detecting a physiological event) may be trained to establish a correspondence between EGMs collected from a population and an assessment of device operating status, such as a change in position, posture, or orientation of the AMD, or a change in an device-tissue interface the AMD. Ambulatory EGMs received from a subject can be applied to the trained model, and an operating status of the AMD can be detected at 530, such as using the device operating state detector 326.

At 540, the estimated physiological parameter or the detected physiological event can be provided to a user or a process, such as to a display or one or more other user interface. The output may include recommendations or alerts to clinicians of any adverse events. The estimated physiological parameter, the detected physiological event, or the detected device operating status may alternatively or additionally to be used to control, adjust, or cease a therapy of a medical device, a drug delivery system, etc. In some examples, Additionally or alternatively, when the estimated physiological parameter satisfies a specific condition, an application-specific sensor can be triggered to directly measure a physiological parameter. The direct measurements of physiological parameters can be used to calibrate the process of estimating physiological parameters or detecting physiological events or device operating status. In some examples, the direct measurements of physiological parameters can be used for adjusting the trained ML model, or in a process of re-training the ML model.

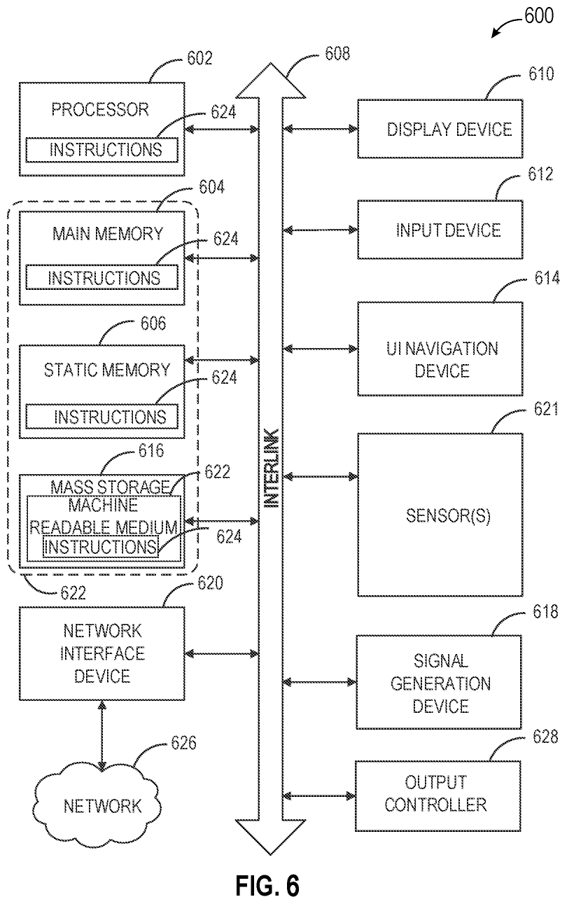

FIG. 6 illustrates generally a block diagram of an example machine 600 upon which any one or more of the techniques (e.g., methodologies) discussed herein may perform. Portions of this description may apply to the computing framework of various portions of the AI-based physiological event detection system 300.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine-readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine-readable media.

While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine-readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802. 11 family of standards known as WiFi®, IEEE 802. 16 family of standards known as WiMax®), IEEE 802. 15. 4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system for detecting physiological events in a subject, the system comprising:
a computing device configured to:
receive ambulatory electrograms of the subject collected by an ambulatory medical device (AMD);
determine or confirm a physical characteristic of at least one sensor using the ambulatory electrograms,; and
detect a physiological event or estimate a physiological parameter in the subject using the ambulatory electrograms and the determined or confirmed physical characteristic of the at least one sensor; and
an output unit configured to output the estimated physiological parameter or the detected physiological event to a user or a process.

2. The system of claim 1, wherein the at least one sensor is configured to sense from the subject physiological information different than the ambulatory electrogram,
wherein to detect the physiological event, the computing device is configured to apply the received ambulatory electrograms to a trained machine learning model to estimate the physiological parameter or to detect the physiological event in the subject using the ambulatory electrograms and the physiologic information sensed by the at least one sensor, and
wherein the physical characteristic is a sensor type or a form factor of the at least one sensor.

3. The system of claim 2, wherein the computing device includes a training module configured to generate the trained machine learning model, including:
constructing a training dataset including ambulatory electrograms collected from a patient population and assessments of physiological parameters or physiological events in the patient population; and
training a machine learning model using the constructed training dataset until a convergence or training slopping criterion is satisfied, the trained machine learning model representing a correspondence between the ambulatory electrograms of the patient population and the physiological parameters or the physiological events in the patient population.

4. The system of claim 3, wherein the training module is configured to train the machine learning model using a deep learning algorithm comprising a deep neural network.

5. The system of claim 2, wherein the computing device is configured to apply the received ambulatory electrograms to the trained machine learning model to estimate the physiological parameter including at least one of:
a cardiac parameter;
a respiratory parameter;
a circulating biomarker; or
a systemic or local fluid status.

6. The system of claim 2, wherein the computing device is configured to apply the received ambulatory electrograms to the trained machine learning model to detect the physiological event including at least one of:
a cardiac arrhythmia;
a worsening heart failure event;
a heart failure comorbidity condition;
a neurological condition; or
a response to medication.

7. The system of claim 2, wherein the computing device is further configured to apply the received ambulatory electrograms to the trained machine learning model to detect an operating status of the AMD.

8. The system of claim 7, wherein the operating status of the AMD indicates at least one of:
a change in position, posture, or orientation of the AMD; or
a change in an device-tissue interface the AMD.

9. The system of claim 1, comprising the AMD configured to collect the ambulatory electrograms of the subject continuously or periodically via one or more attachable or implantable electrodes, the AMD including at least one of:
an insertable cardiac monitor;
a subcutaneous implantable cardioverter-defibrillator; or
a wearable or holdable cardiac monitor.

10. The system of claim 1, wherein the AMD includes a therapy circuit configured to initiate or adjust a therapy to the subject based on the estimated physiological parameter or the detected physiological event.

11. The system of claim 2, wherein the computing device is configured to:
apply the received ambulatory electrograms to the trained machine learning model to estimate the physiological parameter; and
in response to the estimated physiological parameter satisfying a condition, trigger at least one of the at least one sensor to directly measure the physiological parameter.

12. The system of claim 11, wherein the computing device includes a calibration circuit configured to adjust the trained machine learning model based at least on the directly measured physiological parameter.

13. The system of claim 1, wherein the computing device is configured to perform parallel computing to estimate multiple physiological parameters or to detect multiple physiological events substantially concurrently.

14. The system of claim 13, wherein the computing device includes multiple processors or a multi-core processor comprising multiple computing units, each of the multiple processors or the multiple computing units configured to apply a portion of the ambulatory electrograms of the subject to a respectively trained machine learning model to estimate a respective physiological parameter or to detect a respective physiological event in the subject.

15. A method for detecting physiological events in a subject, the method comprising:
receiving ambulatory electrograms of the subject collected by an ambulatory medical device (AMD);
determining, via a computing device, a form factor of at least one sensor using the ambulatory electrograms, the at least one sensor configured to sense from the subject physiological information different than the ambulatory electrograms;
serially detecting a physiological event or estimating a physiological parameter in the subject using the ambulatory electrograms and the physiological information sensed by the at least one sensor; and
providing the estimated physiological parameter or the detected physiological event to a user or a process.

16. The method of claim 15, wherein serially detecting the physiological event includes applying the received ambulatory electrograms to a trained machine learning model to estimate the physiological parameter or to detect the physiological event in the subject.

17. The method of claim 16, comprising:
constructing a training dataset including ambulatory electrograms collected from a patient population and assessments of physiological parameters or physiological events in the patient population; and
training a machine learning model using the constructed training dataset until a convergence or training slopping criterion is satisfied, the trained machine learning model representing a correspondence between the ambulatory electrograms of the patient population and the physiological parameters or the physiological events in the patient population.

18. The method of claim 16, further comprising applying the received ambulatory electrograms to the trained machine learning model and detecting an operating status of the AMD.

19. The method of claim 15, further comprising initiating or adjusting a therapy via the AMD to the subject based on the estimated physiological parameter or the detected physiological event.

20. The method of claim 16, further comprising, in response to the estimated physiological parameter satisfying a condition:
triggering direct measurement of the physiological parameter using at least one of the at least one sensor; and
adjusting the trained machine learning model based at least on the direct measurement of the physiological parameter.
